(19) United States
(12) Patent Application Publication
(10) Pub. No.: US 2020/0055004 A1
(43) Pub. Date: Feb. 20, 2020

(54) EXTRACORPOREAL BLOOD CIRCUIT

(71) Applicant: GAMBRO LUNDIA AB, Lund (SE)

(72) Inventor: Kai HARENSKI, Munich (DE)

(21) Appl. No.: 16/603,695

(22) PCT Filed: Apr. 10, 2018
(86) PCT No.: PCT/EP2018/059098

§ 371 Date: Oct. 8, 2019

(30) Foreign Application Priority Data
Apr. 10, 2017 (EP) 17165657.2

(51) Int. Cl. B01D 69/14; B01D 63/02; B01D 71/42; B01D 71/60; B01D 71/82; A61M 1/16; A61M 1/36

(52) U.S. Cl. CPC B01D 69/147; B01D 63/02; B01D 71/42; B01D 71/60; B01D 2325/16; A61M 1/1621; A61M 1/1698; A61M 1/3666; B01D 2325/12; B01D 71/82

(57) ABSTRACT

The present disclosure relates to extracorporeal blood circuits used for gas exchange in blood, in particular circuits for cardiopulmonary bypass.

### TECHNICAL FIELD

The present disclosure relates to extracorporeal blood circuits used for gas exchange in blood, in particular circuits for cardiopulmonary bypass.

### DESCRIPTION OF THE RELATED ART

Cardiopulmonary bypass (CPB) is commonly used in various surgical procedures performed on the heart or the lung of a patient. Examples include coronary artery bypass surgery, cardiac valve repair and/or replacement, repair of large septal defects, repair and/or palliation of congenital heart defects, transplantation, pulmonary thromboendarterectomy, or pulmonary thrombectomy. A machine pumps the blood, and, using an oxygenator, allows red blood cells to pick up oxygen, as well as allowing carbon dioxide levels to decrease. This mimics the function of the heart and the lungs, respectively. CPB mechanically circulates and oxygenates blood for the body while bypassing the heart and lungs. It uses a heart-lung machine (HLM) to maintain perfusion to other body organs and tissues while the surgeon works in a bloodless surgical field.

During the surgical procedure, excessive water has to be removed from the patient, e.g., by ultrafiltration. Hemofilters are commonly used for this purpose on heart-lung machines.

Extracorporeal membrane oxygenation (ECMO), also known as extracorporeal life support (ECLS), is an extracorporeal technique of providing both cardiac and respiratory support to persons whose heart and lungs are unable to provide an adequate amount of gas exchange to sustain life. ECMO works by removing blood from the person's body and artificially removing the carbon dioxide and oxygenating red blood cells. In veno-arterial (VA) ECMO, this blood is returned to the arterial system and in veno-venous (VV) ECMO the blood is returned to the venous system.

During extracorporeal blood circulation, inflammatory mediators such as cytokines and endotoxins are produced that can lead to a systemic inflammatory response. Multiple measures have been developed over the years to reduce inflammatory release. More recently, it has been proposed to integrate an adsorptive column capturing cytokines into the extracorporeal circuit.

### SUMMARY

The present disclosure provides an extracorporeal circuit in which ultrafiltration and the adsorption of inflammatory mediators is combined in a single device.

### DETAILED DESCRIPTION

An extracorporeal blood circuit is provided which comprises a gas exchange device and a diffusion- and/or filtration device. The diffusion- and/or filtration device comprises semipermeable membranes comprising i) a copolymer of acrylonitrile and sodium methallyl sulfonate, ii) a polyethyleneimine, and iii) heparin. In one embodiment of the extracorporeal circuit of the present disclosure, the diffusion- and/or filtration device is a capillary dialyzer comprising a plurality of semipermeable hollow fiber membranes, the membranes comprising i) a copolymer of acrylonitrile and sodium methallyl sulfonate; ii) a polyethyleneimine; and iii) heparin.

The blood compartment of the capillary dialyzer, which comprises the lumen of the hollow fiber membranes, is connected to the extracorporeal blood circuit. In other words, during operation of the extracorporeal blood circuit, the blood flow in the extracorporeal blood circuit passes through the blood compartment of the capillary dialyzer and thus through the lumen of the hollow fiber membranes, as is the customary setup for a dialyzer.

Diffusion- and/or filtration devices comprising semipermeable membranes comprising i) a copolymer of acrylonitrile and sodium methallyl sulfonate, ii) a polyethyleneimine, and iii) heparin are known in the art.

US 2003/0021826 A1 discloses binding an anticoagulation agent in a stable manner to the surface of semi-permeable membranes essentially comprised of a copolymer of acrylonitrile and at least one anionic and anionizable monomer. A semi-permeable composite membrane is disclosed which comprises a semi-permeable support membrane and an anticoagulation agent suitable for the treatment of blood or plasma by extracorporeal circulation, said semi-permeable support membrane being essentially constituted by a polyacrylonitrile carrying anionic or anionizable groups; the surface of the semipermeable support membrane intended to be brought into contact with the blood or plasma is coated in succession with a cationic polymer carrying cationic groups which can form an ionic bond with anionic or anionizable groups of polyacrylonitrile, the cationic polymer (for example ample polyethyleneimine, PEI) comprising chains of a size which is sufficient not to traverse the semi-permeable support membrane, and an anticoagulation agent carrying anionic groups which are capable of forming an ionic bond with cationic groups of said cationic polymer (for example heparin).

WO 2007/148147 A1 describes the use, on a membrane preferably based on a copolymer of acrylonitrile and sodium methallyl sulfonate, of a solution of a polymer carrying anionic or anionizable groups in the colloidal form and in an acidic medium, in particular by mixing, for example, a solution of polymer carrying anionic or anionizable groups with a solution of organic polyacid in a specific proportion with respect to said polymer, which results in an increase in both the quantity of polymer grafted to the surface of the membrane and the availability of free cationic or cationizable groups at the surface of this membrane coating. The membrane described allows a large quantity of compounds carrying anionic or anionizable groups to be bound. It is suggested for the treatment of septic syndrome, in particular for adsorbing endotoxins contained in the biological fluid, for purifying certain molecules contained in the blood or the plasma by extracorporeal circulation and for reducing systemic anticoagulation in a patient during an extracorporeal blood or plasma treatment. A method for preparing the membrane is also described in WO 2007/148147 A1.

The diffusion- and/or filtration device removes excessive water from the patient by ultrafiltration and also adsorbs inflammatory mediators from the blood of the patient. Examples of suitable diffusion- and/or filtration devices include commercial capillary dialyzers available from Gambro Lundia AB under the trade names oXiris® and Evodial®.

One example for a current product comprising a AN69 type membrane is the Evodial dialyzer, which is a hemodialyzer equipped with a heparin-grafted acrylonitrile based membrane such as described in the aforementioned WO 2007/148147 A1 (the so-called HeprAN membrane). The Evodial membrane is characterized also in that the charged surface, originating from anionic sulfonate groups, is neutralized by the polycationic biopolymer polyethyleneimine. The surface treatment also allows the almost irreversible fixing of said heparin through very strong ionic binding between the negative charges of heparin and the free positive charges of the cationic polymer. Membranes having the ability to immobilize heparin are highly desirable as it further reduces the need of systemic doses of heparin.

In one embodiment of the extracorporeal blood circuit, the membranes of the diffusion- and/or filtration device comprise polyethyleneimine in an amount of from 10 to 100 mg, for instance, from 25 to 50 mg per m² of membrane surface area, and from 1,500 to 10,000 UI, for instance, 3,000 to 6,000 UI, e.g. 4,500±1,500 UI, of heparin per m² of membrane surface area.

In one embodiment, the diffusion- and/or filtration device comprises hollow fiber membranes having an inner diameter in the range of from 180 to 260 μm, e.g. 210 μm, or 240 μm. In one embodiment, the wall strength of the hollow fiber membranes is in the range of from 30 to 60 μm, e.g., 40 to 50 μm. In one embodiment, the overall surface area of the membranes in the diffusion- and/or filtration device is in the range of from 1 to 3 m², for instance, 1.0 to 2.2 m².

In one embodiment, the hollow fiber membranes in the diffusion- and/or filtration device show sieving coefficients, measured at 37° C. in bovine plasma having a protein content of 60 g/l, of >0.95 for inulin; >0.55 for myoglobin; and <0.01 for albumin.

The membranes in the diffusion- and/or filtration device have a remarkable ability to immobilize inflammatory mediators and endotoxins to their surface by adsorption. In one embodiment, the clearance rate for IL-6 in HDF mode with Q_{B}=400 ml/min, Q_{D}=700 ml/min and UF=100 ml/min is in the range of from 20 to 40 ml/min.

The extracorporeal blood circuit of the present disclosure also comprises a gas exchange device. The gas exchange device performs gas exchange in the blood of the patient, i.e., it removes carbon dioxide from the blood and/or oxygenates the blood. In one embodiment of the extracorporeal circuit, the gas exchange device is a membrane oxygenator. Membrane oxygenators are known in the art, and a large variety of devices is commercially available. The membrane oxygenator comprises semipermeable polymer membranes. Examples of suitable polymers include polypropylene, polyethylene, poly-4-methylpentene (PMP), polyvinylidene fluoride (PVDF), and polytetrafluoroethylene (PTFE). In another embodiment, the gas exchange device is a bubble oxygenator. Bubble oxygenators also are known in the art, and commercially available.

In one embodiment, the extracorporeal blood circuit of the present disclosure is a cardiopulmonary bypass (CPB). In one embodiment, the cardiopulmonary bypass is a venoarterial bypass. In another embodiment, the cardiopulmonary bypass is a veno-venous bypass. In one embodiment, the extracorporeal blood circuit comprises a heart-lung machine (HLM). In another embodiment, the extracorporeal blood circuit is configured for extracorporeal membrane oxygenation (ECMO). In one embodiment, the extracorporeal blood circuit is configured for veno-arterial (VA) ECMO. In another embodiment, the extracorporeal blood circuit is configured for veno-venous (VV) ECMO.

The present disclosure also concerns the use of a diffusion- and/or filtration device comprising semipermeable membranes in cardiopulmonary bypass or ECMO. The membranes comprise i) a copolymer of acrylonitrile and sodium methallyl sulfonate; ii) a polyethyleneimine; and iii) heparin. In one embodiment, the diffusion- and/or filtration device is a capillary dialyzer comprising a plurality of semipermeable hollow fiber membranes. In one embodiment, the cardiopulmonary bypass comprises a membrane oxygenator. In a further embodiment, the cardiopulmonary bypass comprises a heart-lung machine.

When blood from a patient passes through the extracorporeal circuit of the present disclosure, the gas exchange device removes carbon dioxide from the blood and/or oxygenates it, i.e., replenishes it with oxygen. Inflammatory mediators and endotoxins are adsorbed by the diffusion- and/or filtration device; and water is withdrawn from the bloodstream by ultrafiltration. It is an advantage of the extracorporeal circuit of the present disclosure that removal of inflammatory mediators and endotoxins as well as removal of excess water from the patient are accomplished using a single diffusion- and/or filtration device. This greatly simplifies the assembly and operation of the extracorporeal circuit. It is hypothesized that in comparison to a cardiopulmonary bypass which only features a hemofilter for removing excess water, the additional removal of inflammatory mediators and endotoxins positively affects the outcome of surgical procedures requiring CPB.

It will be readily apparent to one skilled in the art that various substitutions and modifications may be made to the invention disclosed herein without departing from the scope and spirit of the invention.

The present invention is illustrated by way of non-limiting examples which are further detailed in the Figures accompanying them in order to further facilitate the understanding of the invention.

### EXAMPLES

Removal of inflammatory mediators and endotoxins by a capillary dialyzer having an effective surface area of 1.5 m² (oXiris®, Gambro Lundia AB) was tested. The dialyzer comprises hollow fiber membranes comprising a copolymer of acrylonitrile and sodium methallyl sulfonate. The surface of the fibers comprises 30 mg/m² of high MW polyethyleneimine (PEI) and is grafted with 4,500±1,500 UI/m² of heparin. The fibers have an inner diameter of 240 μm and a wall strength of 40 μm.

Interleukin-10 (IL-10; MW=18 kDa), Interleukin 6 (IL-6; MW=26 kDa), High-mobility group box 1 (HMGB-1; MW=30 kDa) and Tumor necrosis factor alpha (TNF-a; MW=51 kDa) were used as cytokines. Lipopolysaccharide (LPS; MW>100 kDa) was used as endotoxin.

The in vitro experiments were conducted using a pool of 500 ml of frozen human plasma with a protein content of 60±5 g/l from healthy volunteers stabilized with a LPS theoretical concentration of [25-75] EU/mL. Spiking of IL-10, IL-6, HMGB-1 and TNF-a cytokines at a respective concentration of 500 pg/mL, 1,500 pg/mL, 30 ng/mL and 250 pg/mL was performed prior to the start of circulation.

A number of n=3 samples was selected (including n=3 multiple batches), which has been found adequate to demonstrate a systematic removal mechanism and considering that clinical outcomes related to endotoxin/cytokine removal may significantly differ depending on the patient pathological conditions (no direct clinical correlation between the foreseen cytokine adsorbed quantity and the plasma final circulating cytokine level). The circulation conditions are representative of standard CRRT practices.

Prior to circulation of the plasma pool, the blood compartment of the dialyzer was washed with 1.5 L of heparinized (5 UI/mL) saline solution at 150 ml/min. Additional 500 ml of the solution were used to rinse the dialysate compartment by ultrafiltration.

The plasma pool was circulated in a closed loop for 120 min at 150 ml/min. Sampling was performed during the circulation at baseline and after t=5, t=10, t=30, t=60 and t=120 min directly from the plasma pool.

Concentration of cytokines was measured in duplicate by enzyme-linked immunosorbent assay (ELISA) utilizing matched antibody pairs and recombinant (Quantikine R&D system, France; and IBL international, Germany for human HMGB-1). LPS concentration was measured in duplicate by limulus amoebocyte lysate (LAL) chromogenic method (K-QCL Lonza assay).

Removal Rate specifications at the end of the circulation (t=120 min) are calculated for each mediator using:

RR_{(t=120 min)}=[1−C_{(t=120 min)}/C_{(t=0 min)}]×100

with

C_{(t=0 min)}: initial concentration in the plasma pool (baseline sample)

C_{(t=120 min)}: final concentration in the plasma pool at t=120 min

The results are summarized in Table 1.

TABLE 1
Mean calculated removal rate at t = 120 min for IL-10,
IL-6, HMGB-1, TNF-a cytokines and LPS

| | Cytokine adsorption RR (%) | | | | LPS adsorption |
|---|---|---|---|---|---|
| | IL-10 | IL-6 | HMGB-1 | TNF-α | RR (%) |
| Batch 1 | 94.1 | 82.9 | 93.0 | 84.5 | 73.6 |
| Batch 2 | 97.9 | 82.0 | 92.7 | 77.4 | 77.8 |
| Batch 3 | 97.0 | 82.8 | 99.3 | 60.2 | 82.0 |
| Mean (n = 3) | 96.3 | 82.6 | 95.0 | 74.0 | 77.8 |
| SD | 2.0 | 0.5 | 3.7 | 12.5 | 4.2 |
| CV (%) | 2.1 | 0.6 | 3.9 | 16.9 | 5.4 |

*calculated at t = 120 min

The in vitro experiments confirm the high affinity of the oXiris® membrane to the different cytokines challenged (IL-10/IL-6/HMGB-1 and TNF-a) as well as to the endotoxin LPS. Those observations appear consistent with available clinical evidence, suggesting the capability of the device for selective elimination of inflammatory mediators and associated improved patient condition (improved SOFA score and cardiovascular function).

1. An extracorporeal blood circuit comprising
a) a gas exchange device; and
b) a capillary dialyzer comprising a plurality of semipermeable hollow fiber membranes, wherein the membranes comprise i) a copolymer of acrylonitrile and sodium methallyl sulfonate, ii) a polyethyleneimine, and iii) heparin, and
wherein the blood compartment of the dialyzer comprising the lumen of the semipermeable hollow fiber membranes is connected to the extracorporeal blood circuit.

2. The extracorporeal blood circuit of claim 1, wherein the gas exchange device is a membrane oxygenator.

3. The extracorporeal blood circuit of claim 1, wherein the extracorporeal blood circuit is a cardiopulmonary bypass (CPB).

4. The extracorporeal blood circuit of claim 3, wherein the blood circuit comprises a heart-lung machine (HLM).

5. The extracorporeal blood circuit of claim 1, wherein the extracorporeal blood circuit is configured for extracorporeal membrane oxygenation (ECMO).

6. The extracorporeal blood circuit of claim 3, wherein the cardiopulmonary bypass is a veno-arterial bypass.

7. The extracorporeal blood circuit of claim 3, wherein the cardiopulmonary bypass is a veno-venous bypass.

8.-11. (canceled)

12. A method of treating a subject via an extracorporeal blood circuit, said method comprising the step of administering a diffusion device to the subject,
wherein the diffusion device comprises semipermeable membranes comprising i) a copolymer of acrylonitrile and sodium methallyl sulfonate; ii) a polyethyleneimine; and iii) heparin, in a cardiopulmonary bypass.

13. The method of claim 12, wherein the diffusion device is a capillary dialyzer.

14. The method of claim 12, wherein the cardiopulmonary bypass comprises a membrane oxygenator.

15. The method of claim 12, wherein the cardiopulmonary bypass comprises a heart-lung machine.

16. The method of claim 12, wherein the cardiopulmonary bypass is a veno-arterial bypass.

17. The method of claim 12, wherein the cardiopulmonary bypass is a veno-venous bypass.

18. A method of treating a subject via an extracorporeal blood circuit, said method comprising the step of administering a filtration device to the subject,
wherein the filtration device comprises semipermeable membranes comprising i) a copolymer of acrylonitrile and sodium methallyl sulfonate; ii) a polyethyleneimine; and iii) heparin, in a cardiopulmonary bypass.

19. The method of claim 12, wherein the filtration device is a capillary dialyzer.

20. The method of claim 12, wherein the cardiopulmonary bypass comprises a membrane oxygenator.

21. The method of claim 12, wherein the cardiopulmonary bypass comprises a heart-lung machine.

22. The method of claim 12, wherein the cardiopulmonary bypass is a veno-arterial bypass.

23. The method of claim 12, wherein the cardiopulmonary bypass is a veno-venous bypass.
